(19) United States
(12) Patent Application Publication
(10) Pub. No.: US 2013/0210654 A1
(43) Pub. Date: Aug. 15, 2013

(54) Multiplex Immune Effector Molecule Assay

(71) Applicants: Jane Christopher-Hennings, Arlington, SD (US); Steven Lawson, Baltic, SD (US); Eric Nelson, Volga, SD (US); Ying Fang, Brookings, SD (US); Joan K. Lunney, Bethesda, MD (US)

(72) Inventors: Jane Christopher-Hennings, Arlington, SD (US); Steven Lawson, Baltic, SD (US); Eric Nelson, Volga, SD (US); Ying Fang, Brookings, SD (US); Joan K. Lunney, Bethesda, MD (US)

(73) Assignee: SOUTH DAKOTA STATE UNIVERSITY, Brookings, SD (US)

(21) Appl. No.: 13/703,025

(22) PCT Filed: Jun. 9, 2011
(86) PCT No.: PCT/US11/39874

§ 371 Date: Apr. 21, 2013

(51) Int. Cl. G01N 33/68

(52) U.S. Cl. CPC G01N 33/6869; G01N 33/6863; G01N 33/6866
USPC 506/9; 436/501

(57) ABSTRACT

Methods for detecting at least seven cytokines in a porcine biological sample are provided. Also provided are multiplex assay kits that allow for the detection and quantification of the cytokines in a single reaction mixture. Use of the methods and kits for diagnosis, prognosis, and monitoring of immunity is also contemplated.

### CROSS-REFERENCE TO RELATED APPLICATION

This Application claims priority to U.S. Provisional Patent Application Ser. No. 61/353,537 filed Jun. 10, 2010, which application is hereby incorporated by reference in its entirety.

### STATEMENT REGARDING FEDERALLY SPONSORED RESEARCH OR DEVELOPMENT

This invention was made with U.S. Government support from the following agency: XXX. The U.S. Government has certain rights in this invention.

### TECHNICAL FIELD

The present disclosure relates to multiplex assays to measure concentrations of immune effector molecules in a biological sample. More particularly, the embodiments of the present disclosure encompass assays to measure cytokines in porcine serum. A method of using the multiplex assay to measure swine cytokine expression following vaccination against porcine reproductive and respiratory syndrome virus is also contemplated.

### BACKGROUND

Measurement of immune response is important for immune diagnosis of many infections and autoimmune diseases, as a marker for immunocompetence, and for detection of immune response to endogenous and exogenenous antigens, i.e. vaccines. Generally, an immune response is measured by determining the concentration or expression of certain immune effector molecules, such as cytokines.

In certain swine diseases, such as porcine reproductive and respiratory syndrome virus (PRRSV), immune effector molecule expression levels change following natural infection. Expression levels also change following vaccination, thus expression levels of certain immune effector molecules subsequent to vaccination can be used as a predictor of immune response post vaccination.

Immune effector molecules such as cytokines may be measured; however, few standardized assays are available for determining immune effector molecule concentrations in swine. Currently available commercial assays require that analysis be performed individually for each immune effector molecule of interest. This analysis is not only time consuming, but it also requires large sample sizes and significant cost.

The development of a unified or simultaneous immune effector molecule assay has thus far been discouraged by the technology required to perform multi-analyses and by differences among the properties of the particular markers used to measure immune effector molecules. For example, some immune effector molecules are present in lower concentrations than others and therefore require assays of greater sensitivity. Furthermore, the chemistries of the immunoassays differ from one immune effector molecule to the next, and different reagents are added at different times. It is a challenge to accommodate these differences and produce an assay that can provide individual values for each of the immune effector molecules and yet be performed in a single reaction mixture.

### SUMMARY

Disclosed are methods of detecting the presence or concentration of a plurality of immune effector molecules in a biological sample. Generally, the biological sample is porcine. In some embodiments, at least seven different immune effector molecules will be measured. These immune effector molecules may be cytokines such as IL-1β, IL-4, IL-8, IL-10, IL-12, IFN-α, IFN-γ, and TNF-α.

To measure the immune effector molecules, the biological sample is incubated under suitable conditions with capture and detection particles. The capture particle, immune effector molecule, and detection particle form a complex which allows a measurement of the presence or concentration of the immune effector molecule in the biological sample. The biological sample is incubated with the capture particle and the detection particle sequentially in many embodiments.

The capture and detection particles may be monoclonal antibodies which are specific for a particular immune effector molecule. In most embodiments, each capture detection particle can be uniquely identified and is bound to a solid phase support such as a microsphere during the steps of the method. The detection particles are commonly biotinylated such that they can be detected using known biotin-avidin detection methods.

A method of determining the immunity status of a subject is also contemplated. In one embodiment, the immunity status is the immunity status of the subject to porcine reproductive and respiratory syndrome virus (PRRSV). The immunity status can be determined through measurement the presence or concentration of a plurality of immune effector molecules in a biological sample. In exemplary embodiments, the measurement is done (a) prior to vaccination of a subject, (b) subsequent to vaccination of a subject, or (c) both prior to and subsequent to vaccination.

Also disclosed are kits for detecting the presence or concentration of a plurality of immune effector molecules in a biological sample. The kits generally contain capture particles and detection particles as well as buffers.

### BRIEF DESCRIPTION OF THE DRAWINGS

FIG. 1 demonstrates the standard curves generated in the multiplex assays. Comparing the standard curve values for each cytokine in the singleplex vs. multiplex format, the coefficient determinations (R2) were between 0.95 to 1.0 for all 9 cytokines (IL-1β (0.998); IL-4 (1.0); IL-6 (0.990); IL-8 (0.950); IL-10 (0.996); IL-12 (0.990); IFN-α (0.986); IFN-γ (1.0); TNF-α (0.951)). Intra-assay variability of the 9-plex cytokine assay ranged between 3-18% with a mean CV of 10% and inter-assay assay variability ranged between 7.5-18% with a mean of 11.3%.

FIG. 2(a-g) shows cytokine serum concentrations (pg/ml) from pigs given MLV (n=10), KV/ADJ (n=10) or no vaccine (controls) (n=5) at 28 and 32 day post vaccination which corresponds to 0 and 4 day post challenge, respectively. Different letters indicate statistical differences (P≦0.05).

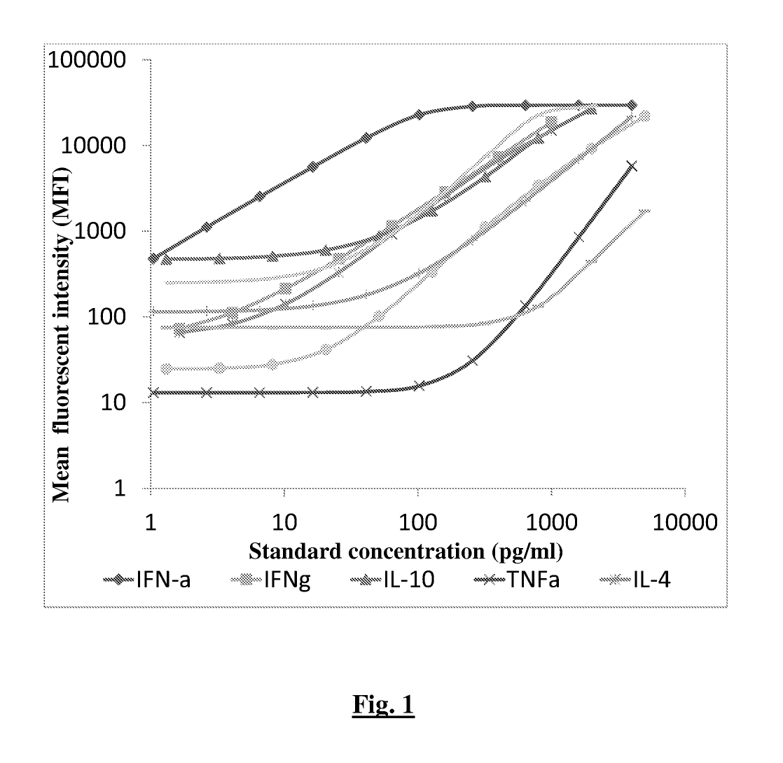

### DETAILED DESCRIPTION

For describing invention herein, the exemplary embodiments in detail, it is to be understood that the embodiments are not limited to particular compositions or methods, as the compositions and methods can, of course, vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which an embodiment pertains. Many methods and compositions similar, modified, or equivalent to those described herein can be used in the practice of the current embodiments without undue experimentation.

As used in this specification and the appended claims, the singular forms “a,” “an” and “the” can include plural referents unless the content clearly indicates otherwise. Thus, for example, reference to “a cytokine” can include a combination of two or more cytokines. The term “or” is generally employed to include “and/or,” unless the content clearly dictates otherwise.

As used herein, “about,” “approximately,” “substantially,” and “significantly” will be understood by person of ordinary skill in the art and will vary in some extent depending on the context in which they are used. If there are uses of the term which are not clear to persons of ordinary skill in the art given the context in which it is used, “about” and “approximately” will mean plus or minus ≦10% of particular term and “substantially” and “significantly” will mean plus or minus >10% of the particular term.

Units, prefixes, and symbols may be denoted in their SI accepted form. Numeric ranges recited within the specification are inclusive of the numbers defining the range and include each integer within the defined range.

The current disclosure provides an assay for simultaneously measuring immune effector molecules in a biological sample taken from subject. As used herein, simultaneous or simultaneously means assaying all of the immune effector molecules of interest at the same time. To be assayed “simultaneously” the different immune effector molecules will be assayed using a single vessel and the same incubation and washing steps (although as is understood, certain reagents will be unique). An immune effector molecule is a molecule that influences the behavior of a regulatory molecule thereby influencing gene expression of genes related to the immune system. Example immune effector molecules include cytokines. The multiplex assay is based on measuring immune effector molecule production by cells of the immune system in response to antigenic stimulation. The immune effector molecules maybe detected using specific capture and detection particles such as antibodies specific for the immune effector molecules.

The disclosed methods and kits have a number of potential uses. They can be used in basic research, i.e. to analyze immune effector molecules in subjects. They can also be used in clinical practice, e.g., for disease diagnosis, for disease prognosis, levels of immunocompentence, and immune responsiveness to endogenous or exogenous antigens, and to monitor subject response to therapeutic or preventative regimens. That is, information on the presence and concentration of immune effector molecules can be used to diagnose a variety of diseases, to predict disease progression, and to monitor response to vaccination and therapies. These methods and kits apply to infectious diseases, as well as other diseases in which differences are exhibited in the pattern of immune effector molecule concentration compared to the normal healthy state.

One aspect disclosed contemplates a method for measuring immune effector molecules in a subject in a multiplex assay, such method comprising collecting a biological sample from the subject and then measuring the presence of, or elevation in the level of specific immune effector molecules as compared to a control sample. In certain embodiments, a baseline measurement, i.e. a measurement prior to infection or immune response is taken from a subject or from a reference animal. This baseline measurement can serve as a control sample. In another embodiment, measurement is taken following natural infection or immunization. The presence or concentration of the immune effector molecule may be indicative of a specific infection. In yet another embodiment, the presence or concentration of an immune effector molecule is indicative of the subject's level of protection against disease following vaccination. Lastly, in still other embodiments, the presence or level of the immune effector molecule is indicative of the capacity of the subject to mount an immune response. A profile of changes in numerous immune effector molecules is also contemplated.

A “subject” includes livestock animals, e.g. sheep, cows, pigs, horses, donkey, goats), and companion animals (e.g. dogs, cats). In one embodiment, the subject is a porcine. The disclosure has applicability in livestock and veterinary applications, and, for example, as used herein can serve as a measurement of immunity following vaccination.

A “multiplex assay” is an assay that simultaneously measures the levels of more than one analyte in a single sample. For example, in the current disclosure, a multiplex assay is an assay capable of measuring at least seven immune effector molecules in one biological sample. An advantage of the multiplex methods and kits, herein disclosed is the small size of biological sample that is required. A second advantage is the ability to detect the presence and concentration of numerous immune effector molecules simultaneously in one reaction container. A third advantage is the ability to quantitate immune effector molecules in a biological sample and a fourth advantage is the ability to directly compare immune effector molecule profiles of normal, healthy and disease-associated or vaccinated subjects.

The disclosed multiplex assays are performed under suitable conditions. As used herein “suitable conditions” are assay conditions which allow detection of at least seven types immune effector molecules in a single reaction, i.e. suitable conditions allow detection of the presence and concentration of a specific immune effector molecule immobilized with a capture particle and a detection particle.

Generally immune effector molecules come from effector cells, which are cells active in antigen disposal by either cell-mediated or humoral immunological responses. The immune effector molecules measured in the methods or assays may be any of a range of molecules produced in response to cell activation or stimulation by an antigen. Specific immune effector molecules include a range of cytokines such as interferons, e.g. Type I and Type II interferons, interleukins (IL), e.g. IL-2, IL-4, IL-10 or IL-12, tumor necrosis factor alpha (TNF-α), a colony stimulating factor (CSF) such as granulocyte (G)-CSF or granulocyte macrophage (GM)-CSF, as well as many others such as complement or components in the complement pathway. Unless explicitly stated differently, as used herein “a” or “an” or “at least” immune effector molecule refers to the subtype of the immune effector molecule and not a single molecule. This is also true when referring to capture particles and detection particles. In one embodiment, the immune effector molecules are IL-1β, IL-4, IL-6, IL-8, IL-10, IL-12, IFN-α, IFN-γ, and TNF-α. Related to this embodiment, another embodiment comprises immune effector molecules of less than the entire list, i.e. one or more of IL-1β, IL-4, IL-6, IL-8, IL-10, IL-12, IFN-α, IFN-γ, and TNF-α.

As disclosed herein, the multiplex assay can be used to measure IL-1β, IL-4, IL-6, IL-8, IL-10, IL-12, IFN, IFN, and TNF simultaneously in the range of pg/ml of biological sample.

Immune effector molecules are measured in a biological sample taken from the subject. Biological samples may be collected from the subject using a variety of methods known in the art and include all clinical samples such as cells, tissues and bodily fluids. Biological samples specifically encompass serum, plasma, adipose interstitial fluid, blister fluid, bronchoalveolar lavage fluid, cerebrospinal fluid, nasal lavage fluid, peritoneal fluid, synovial fluid, colon tissue, kidney tissue, lung tissue, nervous system tissue, spleen tissue, and tissue culture supernatant. In one embodiment, the biological sample is serum. This serum may be isolated from whole blood collected from a porcine.

To assay the immune effector molecules, the biological sample is placed with a capture particle specific for an immune effector molecule under suitable condition. A capture or detection particle “specific for” an immune effector molecule has a higher affinity for that immune effector molecule than for any other material in a biological sample or a mixture. Typically, the capture or detection particle binds the immune effector molecule for which it is specific at least about 10 times more tightly (and preferably at least about 100 times more tightly, at least about 1000 times more tightly, or even at least about 10,000 times more tightly) than any other material in the mixture, e.g., under suitable assay conditions. Capture particles are well known in the art, and their only requirement is that they must not prevent the association of a detection particle. In many embodiments, the capture particle is a capture antibody. A capture antibody is an antibody or antibody fragment capable of specifically binding to a specific immune effector molecule. The capture antibody may be a monoclonal antibody. In other embodiments, the capture antibody is a polyclonal antibody. In certain embodiments, the capture antibody is an IgG fragment. Generally, an “antibody” refers to a polypeptide encoded by an immunoglobulin gene or immunoglobulin genes, or fragments thereof, which specifically bind and recognize an analyte (antigen).

Prior to addition of a biological sample, the capture particle may be immobilized on a solid phase support. Immobilization encompasses non-covalent adsorption as well as covalent attachment. As used herein, a “solid phase support” includes polymers such as nitrocellulose or polystyrene, optionally in the form of a stick, a test strip, a bead, a microsphere bead, or a microtiter tray. A “microsphere” is a small spherical, or roughly spherical, particle. A microsphere typically has a diameter less than about 1000 micrometers (e.g., less than about 100 micrometers, optionally less than about ten micrometers). The microsphere can comprise any of a variety of materials (e.g., silica, polystyrene or another polymer) and can optionally have various surface chemistries (e.g., free carboxylic acid, amine, or hydrazide groups, among many others). In certain embodiments, the solid phase support will be magnetic. Commercially available solid phase supports are well known in the art and the skilled artisan can easily determine an appropriate solid phase support.

Immobilization processes of capture particle to solid phase support are known by the skilled artisan and generally consist of cross-linking covalently binding or physically adsorbing the capture particle to the solid phase support. In one embodiment, the capture particle is bound to the solid phase support in MES in the dark for about three hours at room temperature. For example, monoclonal capture antibodies are bound with a solid phase support of Luminex® polystyrene carboxylated microspheres using a two-step carboiimde coupling procedure. Individual microsphere beads commonly have separate spectral addresses to assist in detection.

The optimal concentration of capture particle to solid phase support can be determined via a titration assay. For example, the appropriate amount of capture monoclonal antibody can range from about 50 μg to about 150 μg of antibody. In one embodiment, the amount of capture monoclonal antibody is 100 μg. Although the optimum amount of capture antibody to solid phase support can be titrated, in an embodiment which uses microsphere beads and monoclonal capture antibody, an optimal concentration of capture particle to solid phase support can be between 16-32 μg/IG/1×10⁶ microsphere beads. Differing solid phase supports as well as differing capture particles will require differing concentrations of capture particle to solid phase support.

For multiplex assays, once capture particle has been immobilized on a solid phase support, different capture particle/solid phase supports may be combined into a capture particle/solid phase support mixture. In one embodiment, the capture particle/solid phase support mixture comprises capture particles for several immune effector molecules. For example, the capture particle mixture may comprise capture particles for up to five, up to six, up to seven, up to eight, or up to nine immune effector molecules.

The capture particle/solid phase support mixture may be washed one or more times to remove unattached capture particle and prepare for the biological sample. In some embodiments, these wash steps take place prior to mixing the capture particle/solid phase supports into a capture particle/solid phase support mixture. The capture particle/solid phase support may be washed 1×, 2×, 3× or more. Washing solutions can include buffers such as PBS-NB. Buffers such as PBS-NB may also be used to block non-specific binding of immune effector molecules to the solid phase support by incubating the immobilized capture particles with the buffer. Blocking incubation times may vary and include up to 20 minutes, up to 30 minutes, up to 1 hour, and up to 24 hours.

Following wash and blocking steps of immobilized capture particle/solid phase support, the immobilized capture particle/solid phase support is resuspended to an appropriate concentration. In many embodiments, the resuspension solution is the same as the wash buffer. Concentrations following resuspension may be from about 1.0×10³ immobilized capture particle/solid phase support per aliquot to about 3.0×10³ immobilized capture particle/solid phase support per aliquot. In one embodiment, the concentration will be about 2.5×10³ immobilized capture particle/solid phase support per aliquot.

An aliquot of the biological sample to be tested is then added to an aliquot of the immobilized capture particle/solid phase support and incubated for a period of time sufficient under suitable conditions to allow immobilization of immune effector molecules in the biological sample to the immobilized capture particle/solid phase support complex. The aliquot of biological sample may be about 25 μl, between about 25 μl and 50 μl, about 50 μl, or more than 50 μl. In one embodiment the biological sample is serum and the amount of biological sample is about 50 μl.

The incubation time of the biological sample with the immobilized capture particle/solid phase support is about 2-120 minutes. In other embodiments, the incubation time is overnight. The temperature at which the incubation takes place can be from about 20° C. to about 40° C. In one embodiment, incubation of the biological sample and immobilized capture particle/solid phase support takes place at room temperature. The incubation may also take place on a shaker. Following an appropriate incubation period, under suitable conditions the immune effector molecule/capture particle/solid phase support complex is washed. In one embodiment, the immune effector molecule/capture particle/solid phase support complex is washed in PBST. The wash steps may be performed 1×, 2×, 3× or more.

Immobilization of the immune effector molecule to the capture particle and exposure of the immune effector molecules to the detection particle can occur simultaneously or sequentially, in various orders. However, generally, the detection particle will be added subsequent to the capture particle. For example, once the immune effector molecule/capture particle/solid phase support complex has been washed, a detection particle specific for an immune effector molecule and capable of producing a detectable signal, is added and incubated to the washed immune effector molecule/capture particle/solid phase support mixture, allowing time sufficient for the formation of a complex of capture particle/solid support/immune effector molecule/detection particle. In many embodiments, the detection particle is a second antibody linked to a reporter. The detection particle may be a monoclonal antibody. The detection particle may also be a polyclonal antibody.

When using a monoclonal antibody as a detection particle, the appropriate concentration of the detection particle may be about 0.5 μg, about 1.0 μg, about 2.0 μg, about 2.5 μg, about 5.0 μg, and about 10.0 μg of detection particle per milliliter of immune effector molecule/capture particle/solid phase support. In one embodiment, the amount is about 0.5 μg/ml. In another embodiment, the amount is about 1.0 μg/ml.

The detection particle may be incubated with the immune effector molecule/capture particle/solid phase support under suitable conditions for a period of about 30 minutes, about 1 hour, about 1.5 hour, or about 2.0 hour. In one embodiment, the detection particle is incubated with the immune effector molecule/capture particle/solid phase support for about 1.5 hour. Following this incubation period the immune effector molecule/capture particle/solid phase support/detection particle complex is usually washed. The immune effector molecule/capture particle/solid phase support/detection particle complex may be washed 1×, 2×, 3× or more in an appropriate buffer. The buffer may be PBST.

The presence and concentration of the immune effector molecule is determined by observation of a signal produced by the detection particle. Detection may either be qualitative, by simple observation of a visible signal, or may be quantitated by comparing with a control sample containing known amounts of immune effector molecule. In many cases, the signal from the detection particle will be from a reporter.

A “reporter” as used in the present specification, is meant a molecule which, by its nature, provides an analytically identifiable signal which allows the detection of detection particle bound to immune effector molecule/capture particle/solid phase support. Detection may be either qualitative or quantitative. The most commonly used reporters in multiplex assays are enzymes, fluorophores or radionuclide containing molecules (i.e. radioisotopes) and chemiluminescent molecules. Examples of applicable reporters are known in the art, such as those demonstrated in U.S. patent application Ser. No. 10/477,571. Reporters may be conjugated to a detection particle by a wide variety of different conjugation techniques, which are readily available to the skilled artisan.

Methods of detection are well known in the art and will depend on the type of detection particle used. Methods of detection are not meant to be limiting and include all methods currently used. A monoclonal antibody detection particle may be biotinylated. So for example, if the detection particle is a biotinylated antibody, the method of detection may be incubation with a strepavidin-R-phycoerthrin solution. The immune effector molecule/capture particle/solid phase support/detection particle complex is incubated with the strepavidin-R-phycoerthirin solution for approximately 30 minutes at room temperature in one embodiment.

In those embodiments where a monoclonal capture particle or detection particle for use in the multiplex assay are not commercially available, monoclonal capture or detection antibodies may be constructed using those methods known in the art.

Although generally, many of the individual disclosed steps have been explained in the art, it is only the current disclosure that has surprisingly and unexpectedly provided for the simultaneous detection of at least seven porcine immune effector molecules. Previous experimentation has been unable to adequately and reliably provide for simultaneous detection of such a large number of immune effector molecules. Advantages to simultaneous detection include lower costs of materials and convenience both in terms of performing assays and collecting samples to assay. In many cases, these advantages can be significant.

Kits (e.g. a kit containing each or some of the components of performing the method) are also disclosed. One general class of embodiments provides a kit for detection of the presence or concentration of a plurality of immune effector molecules in a biological sample. The kit comprises a plurality of capture particles and detection particles packaged in one or more containers. In some embodiments, the kit may also contain solid phase support, wash buffers, incubation buffers, blocking buffers, control immune effector molecules or profiles, and reporters. In one embodiment, the capture particles in the kit will be immobilized to a solid phase support. The kit typically also includes instructions for use of the kit; for example, instructions for immobilizing an immune effector molecule in a biological sample on a capture particle. In one class of embodiments, the kit can be used for diagnosis, prognosis or monitoring of immunity by detection of the presence and concentration of immune effector molecules.

### EXAMPLES

The invention may be further clarified by reference to the following Examples, which serve to exemplify some of the embodiments and not to limit the invention in any way. The experiments were performed using the methodology described below.

I. Covalent Coupling of Capture Antibodies to Carboxylated Microspheres

For each cytokine, the respective capture antibody was covalently coupled to polystyrene, carboxylated microspheres (for example Luminex X-Map™) with separate spectral addresses using a two-step carbodiimide coupling procedure (Table 1). All reactions were performed in 1.5 ml, homopolymer low protein adhesion microcentrifuge tubes. Briefly, 3.1×10⁶ microspheres corresponding to a discrete spectral address were washed twice with 250 μl of activation buffer (0.1M NAH₂PO₄, pH 6.2) and sonicated for 60 seconds after each wash by immersion into a 40 W sonicating water bath. Microspheres were activated for 20 min at room temperature in 500 μl activation buffer containing 2.5 mg of freshly prepared N-hydroxysulfocuccinimide (sulfo-NHS) and 2.5 mg N-(3-dimethylaminopropyl)-N-ethylcarbodiimide (EDC). Activated microspheres were washed twice with coupling buffer (0.5M 2-[N-morpholino]ethanesulfonic acid (MES)), pH 5.0 and sonicated following each wash. Coupling was initiated by the addition of 100 μg of capture mAb into 500 μl fresh MES and allowed to incubate in the dark for 3 hours at room temperature with end-over-end mixing. Coupled microspheres were washed once with 1 ml of PBS+0.05% NaN3+1.0% BSA (PBS-NB) and blocked with an additional 1 ml of PBS-NB for 30 min to reduce non-specific binding. Microspheres were washed an additional two times and re-suspended in PBS-NB, to a final concentration of 2.0×10⁶ antibody-coupled-microspheres/ml in PBS-NB.

TABLE 1
FMIA cytokine capture and detection monoclonal antibodies

| | Capture mAb | | Detection mAb | |
|---|---|---|---|---|
| Cytokine | No. | Clone (source) | No. | Clone (source) |
| IL-1β | 841040 | DY681^{a} (RD) | 841041 | DY681 (RD) |
| IL-4 | 5S12809 | CSC1283^{b} (I) | 18426-31 | 2b2.131 (US) |
| IL-6 | M620 | 5IL6 (PT) | SC80837 | 24D12^{c} (SCB) |
| IL-8 | CXCL8 | 8M6 (S) | MAB5351 | 10510^{c} (RD) |
| IL-10 | ASC0104 | 945A4C437B1 (I) | ASC9109 | 945A1A926C2 |
| | | | | (I) |
| IL-12 | MCA2414Z | G9.2 (S) | BAM9122 | 116211 (RD) |
| IFNα | GTX11408 | G16 (GT) | 27105-1 | F17^{c} (PBL) |
| IFNγ | ASC4934 | A151D5B8 (I) | ASC4839 | A151D13C5 (I) |
| TNF-α | 5S17509 | CSC1753^{b} (I) | 5S17503 | CSC1753^{b} (I) |

^{a}Duoset no. (no clone no.);
^{b}Cytoset no. (no clone no.);
^{c}Commercially available biotinylated mAbs not available, therefore biotinylation procedure performed,
GT: GeneTex, San Antonio, TX;
I: Invitrogen, Carlsbad, CA;
PBL: PBL Biomedical Laboratories, Piscataway, NJ;
PT: ThermoScientific Pierce Protein Research Products, Rockford, IL;;
RD: R & D Systems, Inc., Minneapolis, MN;
SCB: Santa Cruz Biotech, Santa Cruz, CA;
S: AbD Serotec; Raleigh, NC;
US: US Biologicals, Swampscott, MA

II. Coupling Efficiency Determination

A determination of the relative amount of mAb per microsphere was performed by adding 2.5×10³ antibody-coupled microspheres to each column well of a 96-well microtiter filterplate pre-wetted with 20 μl PBS-NB. A solution containing 10 μg/ml of goat anti-mouse strepavidin-R-phycoerythrin (SAPE) (Invitrogen/Molecular Probes, Eugene, Oreg.) was diluted in PBS-NB and serial, log₂ dilutions were performed down separate columns of dilution tubes. Fifty microliters of each titration was added to corresponding wells containing coupled-microspheres and allowed to incubate at room temperature for 1 hour on a plate shaker. Controls included uncoupled microspheres. Microspheres were washed via a vacuum manifold three times with a solution of PBS+0.05% Tween 20 (PBST) then resuspended in 125 μl of PBST and transferred to a 96 well polystyrene optical plate. Coupled microspheres were analyzed through the flow cell of a dual laser Bio-Rad, Bio-Plex 200® instrument analyzed with the Bio-Plex Manager software version 5.0. The median fluorescent intensity (MFI) for 100 microspheres was recorded at each titration point and a five parameter logistic regression curve was generated. Relative coupling efficiencies for each mAb were determined by analyzing the MFI at each dilution point and position under the curve.

Relative microsphere coupling efficiencies were determined by using 10 μg/ml of a goat, anti-mouse IgG phycoerythrin antibody to determine a qualitative amount of each coupled antibody relative to others. The following list shows the MFI of each anti-cytokine microsphere coupled antibody: IFNγ (23,625), IL-10 (24,551), IL-1β (29,520), IL-4 (27,066), TNFα (20,668), IL-8 (14,614), IL-12 (21,848), IFNα (3,756) and IL-6 (30,006).

III. Biotinylation of Detection mAb

Commercially available biotinylated mAbs were obtained for six of the cytokines, but a biotinylation procedure was performed to obtain detection antibodies for IFN-α, IL-6 and IL-8. Briefly, mAbs were dialyzed using a Spectra/Por dialysis membrane, MWCO 10,000 (Spectrum Laboratories, Rancho Dominguez, Calif.) overnight at 4° C. against a 1000× volume of PBS to remove any inhibitory preservatives. Each mAb was then transferred to a microcentrifuge tube and 0.150 mg of biotin-NHS (Calbiochem, La Jolla, Calif.) was added to every milligram of affinity purified antibody in a solution containing PBS+10% DMSO. The solution was incubated in the dark for 4 hours with rotation at room temperature then dialyzed overnight at 4° C. against a 4000× volume of PBS. The conjugated antibody solution was quantified via the Lowry protein method and carrier BSA was added to a final concentration of 10 mg/ml and subsequently aliquoted and stored at −20° C.

IV. Singleplex and Multiplex Assay Procedures

For the “sandwich” FMIA (fluorescent microsphere immunoassay), nine (9) mAbs were used to couple carboxylated microspheres for cytokine protein capture (Table 1). Since serum may shift or reduce the slope of the standard curve compared to buffer alone and to provide a complimentary matrix for standards, dilutions of cytokine standards in pooled porcine sera from clinically healthy pigs was obtained. This pig serum was tested by commercial ELISA and the current FMIA and confirmed that there were no measurable levels of the tested cytokines present. The optimum working dilution of the porcine test sera for dilution of swine cytokine standards was predetermined by titration to give the highest signal to background ratio aside from nonspecific reactions. At a serum dilution of 1:2 in PBS pH 7.2+0.05% NaN₃+1.0% BSA (PBS-NB), a maximum dynamic range for all capture microspheres was attained.

For the FMIA, a 96-well 1.2-μm, hydrophilic membrane, filter plate was blocked for two minutes with 150 μl of PBS-NB then aspirated via a vacuum manifold and wetted with an additional 20 μl of PBS-NB buffer. Cytokine standards (recombinant proteins from commercial sources) were diluted in the above described pooled porcine serum. Next, 50 μl of porcine test serum diluted 1:2 in PBS-NB or diluted standards were added to duplicate wells of the filter plate along with 2.5×10³ of each mAb coupled microspheres in an additional 50 μl buffer. All incubations were performed in the dark by sealing the plate with foil. Plates were incubated at room temperature for 2 hours (incubation times initially tested were 1, 1.5 and 2 h) on a plate shaker rotating at a speed of 750 rpm. Next, the plate was aspirated via vacuum manifold three times and washed with 150 μl of PBST. Then, 50 μl of each anti-cytokine, secondary, biotinylated, mAb was diluted appropriately in PBS-NB and added to the filter plate and incubated in the dark at room temperature for 90 minutes (incubation times initially tested were 0.54, 1. 1.5 and 2 hours), then aspirated and washed three times with PBST. Concentrations were determined by evaluating the sensitivity, fluorescent intensity and slope of 0.5, 1.0, 2.0, 2.5, 5.0 and 10 μg/ml of each biotinylated mAb added to the FMIA. The concentration of each biotinylated mAb was 0.5 μg/ml for IL-10, TNFα, IL-8, IFN-α, IL-12; 1.0 μg/ml for IFN-γ, IL-4; 2.0 μg/ml for IL-1β and 2.5 μg/ml for IL-6. Next, 50 μl of a solution containing 10 μg/ml SAPE in PBS-NB was added to each well and incubated for 30 minutes at room temperature with shaking. The supernatant was then aspirated and washed three times with PBST. Finally, the microspheres were re-suspended in 125 μl of PBST per well and transferred to a clear 96-well polystyrene optical plate. Coupled microspheres were analyzed through the flow cell of a dual laser Bio-Rad, Bio-Plex 200® instrument and analyzed with the Bio-Plex Manager software version 5.0. The MFI for 100 microspheres corresponding to each individual cytokine analyte was recorded for each well. All reported MFI measurements were background corrected (normalized) (F-Fo), where Fo was the background signal determined from the fluorescence measurement of the negative control sample (1:2, control serum: PBS-NB) and F was the MFI for each cytokine containing analyte.

Each cytokine was first tested in singleplex assay using our standard buffer system (PBS-NB) then evaluated in swine serum diluted 1:2 to assess the deviation of calibration slopes between matrices. In addition, each singleplex assay was compared to the 9-plex assay to determine whether there was any cross-reactivity. A correlation coefficient was determined between the singleplex vs. multiplex standard curve values for each cytokine measurement. To further evaluate any cross reactivity between individual capture mAb coupled microspheres and unrelated proteins, each capture mAb coupled microsphere was evaluated with and without the associated cytokine protein and percent cross reactivity was recorded. For example, a MFI level would be obtained with the IL-4 mAb bead was used alone with all cytokines and all biotinylated mAbs and compared to the MFI level without IL-4 protein. In addition, to evaluate any cross reactivity between a specific cytokine and unrelated biotinylated mAbs, all capture mAb coupled beads were used and evaluated against all cytokine proteins with and without the associated biotinylated mAb in a multiplex assay. A percent cross reactivity was recorded between the MFI with and without the associated biotinylated mAb. For these experiments, the upper end of the dynamic range for each cytokine protein was used (e.g. 800-2000 pg/ml).

V. Cytokine ELISA and FMIA Comparisons, Recombinant Protein Standards

Separate swine cytokine ELISA kits were utilized from R & D Systems, Inc., Minneapolis, Minn., for the detection of IL-1β (Duoset, DY681) and IL-12 (Duoset, DY912), and from Invitrogen, Carlsbad, Calif. for the detection of IL-8 (Cytoset, CSC 1223); TNFα (Cytoset, CSC 1753); IFN-γ (Cytoset, CSC 4033) and IL-4 (Cytoset, CSC1283). ELISA procedures were performed as per the manufacturer's instructions. A serial dilution of each recombinant protein supplied with each kit was spiked 1:2 into control pig serum and used for comparisons by determining a correlation coefficient between the ELISA and FMIA. Since ELISA kits were not commercially available for the detection of IFN-α and IL-6, recombinant protein standards for the FMIA were purchased separately from PBL Biomedical Laboratories, Piscataway, N.J. (17100-1) and R & D Systems, Inc. (686-PI/CF), respectively.

In addition, for validation of reactivity of every assay with native as well as recombinant cytokine protein, eleven (11) cell culture supernatants generated with different stimulants (LPS, ionomycin, Concanavalin A) or from experimentally inoculated pigs were examined. These supernatants had been archived and previously tested by various cytokine ELISAs and affirmed that the FMIA detected native cytokine proteins.

V. Cytokine ELISA and FMIA Comparisons

The limits of detection (LOD) and upper ranges of detection in pg/ml were compared between the FMIA and ELISA (Table 2). When serial dilutions of recombinant protein standards were tested by ELISA and FMIA for all cytokines, a correlation coefficient (R2) was also determined as listed on Table 2.

For further verification that native cytokine proteins were detected by the FMIA, cell culture supernatants were used to evaluate the FMIA detection of all of the native cytokine proteins. Seven of 11 cell culture supernatants had detectable IFN-γ from 1-1382 pg/ml; 8 of 11 had detectable IL-4 from 1-90 pg/ml; 9 of 11 had detectable IL-12 from 20-134 pg/ml; 1 of 11 had detectable IL-8 at 465 pg/ml; 2 of 11 had detectable IFN-α at 10 and 21 pg/ml; 8 of 11 had detectable IL-6 from 29-413 pg/ml; 6 of 11 had detectable IL-1β from 11-2463 pg/ml; 4 of 11 had detectable IL-10 from 110-536 pg/ml and 9 of 11 had detectable TNF-α from 1879-6885 pg/ml.

TABLE 2
Correlation coefficients and comparison of the limits of detection
(LOD) and upper range detectable by ELISA and FMIA in pg/ml.

| | FMIA | | ELISA | | |
|---|---|---|---|---|---|
| Cytokine | LOD | Upper Range | LOD | Upper Range | R² |
| IL-1β | 17 | 4000 | 77 | 4000 | 0.998 |
| IL-4 | 1.1 | 1000 | 2.1 | 1000 | 0.994 |
| IL-6 | 129 | 5000 | NA | NA | NA |
| IL-8 | 4.4 | 2000 | 24.4 | 2000 | 0.994 |
| IL-10 | 4.3 | 2000 | 23.5 | 2000 | 0.996 |
| IL-12 | 48 | 5000 | 395 | 5000 | 0.996 |
| IFN-α | 0.36 | 4000 | NA | NA | NA |
| IFN-γ | 3.7 | 1000 | 4.3 | 1000 | 0.986 |
| TNF-α | 126 | 4000 | 10.5 | 4000 | 0.978 |

FMIA: fluorescent microsphere immunoassay;
LOD: limit of detection;
R2: coefficient of determination between ELISA and FMIA;
NA: not applicable (commercial ELISA kits not available)

VII. Animals and Experimental Protocol

The vaccine experimental protocol for this study included testing archived serum from three groups of adult female mixed-breed swine for cytokine analysis. Pigs had been vaccinated with either a MLV PRRSV vaccine, Pyrsvac-183 (Syva Labs, Leon Spain) (n=10); a killed virus vaccine with adjuvant (KV/ADJ) Progressis (Merial Labs, Lyon, France) (n=10), or were non-vaccinated controls (n=5). Vaccine was applied twice at day 0 and 21 days post vaccination (DPV) and pigs in all groups were subsequently challenged at 28 DPV with 105 TCID50 of PRRSV (Lelystad) intranasally. Cytokine analysis on the FMIA was performed on serum from all pigs at 28 and 32 days post vaccination (DPV) which corresponds to 0 and 4 days post challenge (DPC), respectively. These animals had been previously assessed as exhibiting different levels of protective immunity against PRRSV, ranging from a) sterilizing immunity (viremia negative, viral load in tissue negative or low (MLV vaccinated) b) viremia positive, viral load in tissue positive (KV/ADJ and non vaccinated controls).

VIII. Cytokine Analysis at 28 (0 DPC) and 32 DPV (4 DPC)

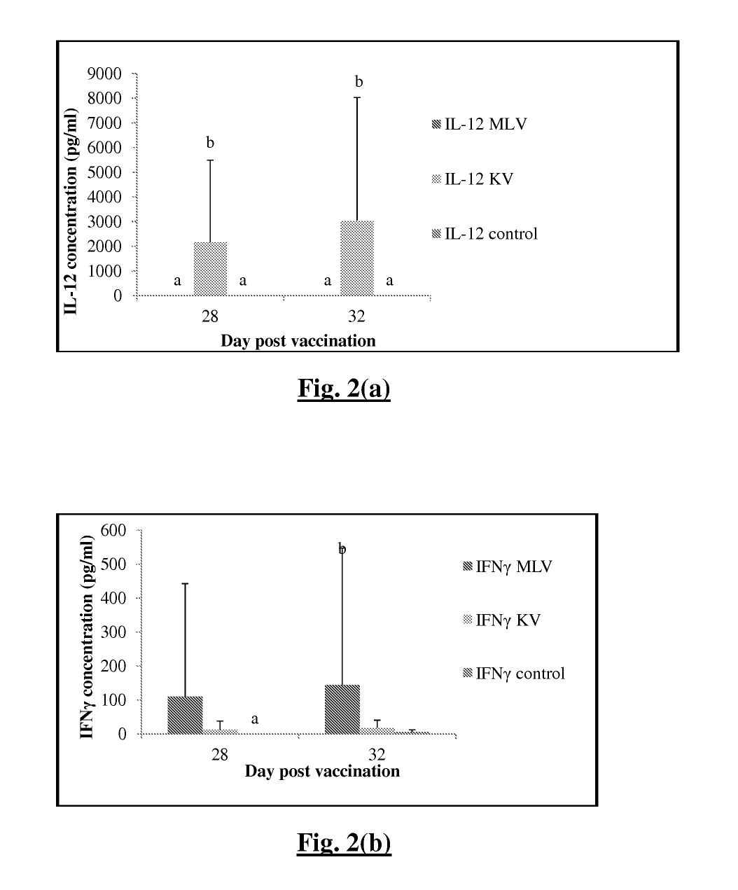

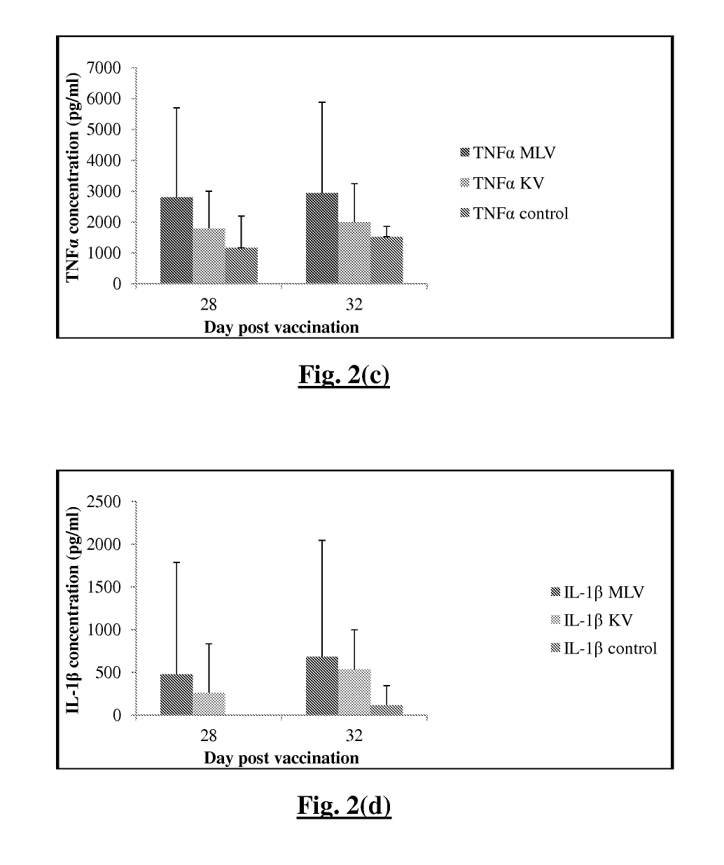

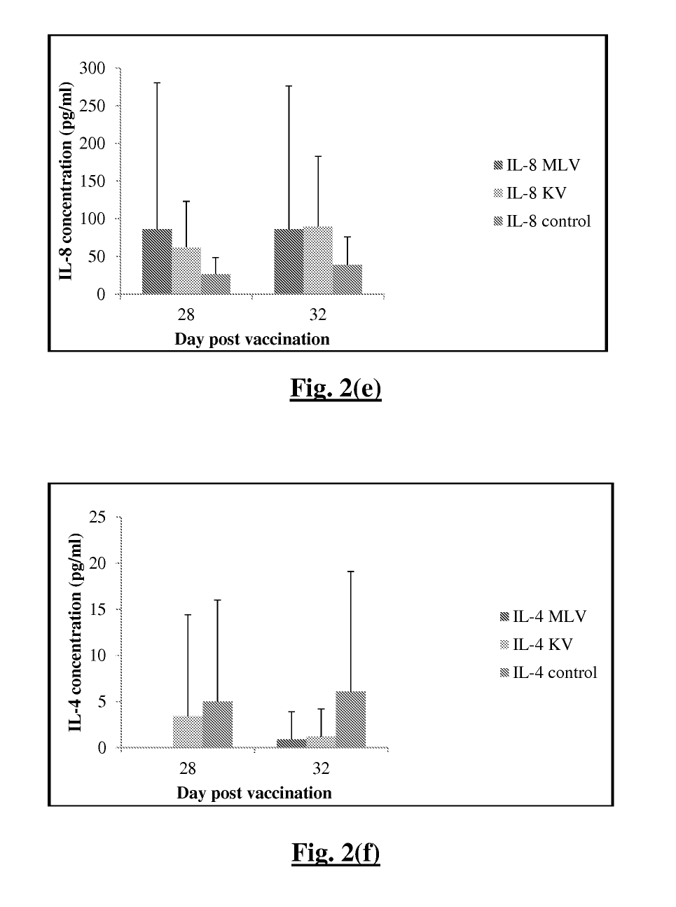

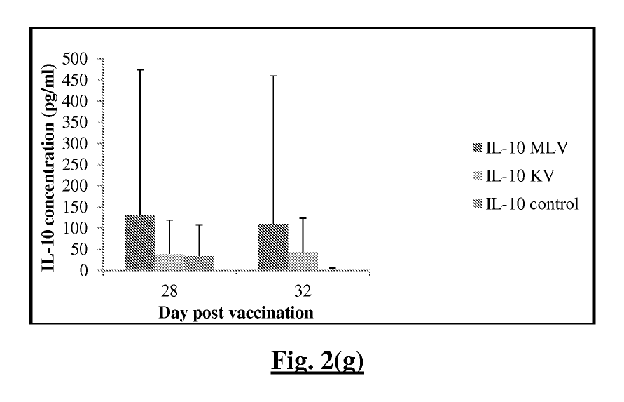

Multiple serum cytokines were altered by the PRRSV vaccination and challenge. A significant increase in the mean IL-12 serum cytokine level was noted for pigs given the KV/ADJ vaccine; no such increase was seen in sera from the MLV pigs at 28 and 32 DPV nor from the control pigs at 0 and 4 DPC (FIG. 2a). Importantly, a predicted protective associated elevation in IFNγ levels was not observed in pigs given the KV/ADJ vaccine for either 28 or 32 DPV whereas it was seen for the MLV pigs (FIG. 2b). A statistically significant difference in IFNγ levels was observed between the control pigs at 28 DPV and pigs given the MLV vaccine at 32 DPV (FIG. 2b). Cytokine levels for IL-1β, IL-4, IL-8, IL-10, IFNα, and TNF-α were measured in the same sera from all pigs in all groups in the same multiplex assays.

IX. Statistics

Before each FMIA run, the multiplex array reader was calibrated against known reporter signal calibrates (CAL2 calibration bead standards), and a dual set of bead spectral address classification calibrates (CL1 target & CL2 target) from Bio-Rad. For each bead class, a total of 100 beads were analyzed using a high RP1 target setting. Samples and standards were measured in duplicate then normalized mean fluorescent intensity (MFI) values were used for the calculation of each immune effector molecule.

FMIA standard curves for all nine cytokines were calculated using a five parameter logistic (5-PL) regression model and cytokine concentrations from experimental samples were obtained via interpolation from best fit regression analysis generated by the Bio-Plex Manager 5.0 software. In addition, the software provides full statistical microsphere data (bead counts, mean, median, % CV, standard deviation & sampling errors). ELISA standard curves were generated via 5-PL regression interpolation using SoftMax Pro 5.0 (Molecular Devices, Sunnyvale, Calif.).

The limits of detection (LOD) for each immune effector molecule was defined as the lowest concentration of each cytokine that can be detected above the lower 5-PL regression asymptote, and was established by analyzing multiple replicates and calculated as the concentration corresponding to the MFI plus 2 standard deviations of the 0 calibrator for each analyte. The analytical range of the assay was assessed from the precision curve and defined as the concentration range in which the CV ([SD/Mean]×100%) was less than 20%.

The determination of intra-as say repeatability was evaluated by analyzing multiple replicates (n=11) of recombinant cytokine standards with known concentration during a single assay run and expressed as the CV of repeated measurements. Interassay variability was studied using 11 different concentrations of standards and analyzed in triplicate over 3 different days (n=9 for each of 11 immune effector molecules) and expressed as the CV of repeated measurements.

The comparison of means between groups of experimental immune effector molecules was performed using GraphPad InStat version 3.06 (GraphPad Software, San Diego, Calif.). Comparison of mean cytokine levels between groups of pigs and days were performed using a non-parametric Kruskal-Wallis statistic. A P≦0.05 was considered statistically significant for all immune effector molecules.

A Pearson's correlation coefficient was determined for singleplex vs. multiplex comparisons and ELISA vs. FMIA comparisons.

Any aspect or design described herein as “exemplary” is not necessarily to be construed as preferred or advantageous over other aspects or designs. Exemplary embodiments may be implemented as a method or composition. The word “exemplary” is used herein to mean serving as an example, instance, or illustration.

All of the references cited herein are incorporated by reference in their entireties.

From the above discussion, one skilled in the art can ascertain the essential characteristics of the invention, and without departing from the spirit and scope thereof, can make various changes and modifications of the embodiments to adapt to various uses and conditions. Thus, various modifications of the embodiments, in addition to those shown and described herein, will be apparent to those skilled in the art from the foregoing description. Such modifications are also intended to fall within the scope of the appended claims.

What is claimed is:

1. A method of detecting the presence or concentration of a plurality of immune effector molecules in a biological sample, comprising:
(a) incubating a biological sample under suitable conditions with (i) at least seven capture particles, wherein each capture particle is specific for an immune effector molecule, further wherein only one capture particle is specific for each immune effector molecule, and (ii) at least seven detection particles specific for the same immune effector molecules as the at least seven capture particles, wherein each detection particle comprises a reporter, further wherein only one detection particle is specific for each immune effector molecule;
(b) forming a complex by (a) immobilizing each immune effector molecule to a capture particle specific for the immune effector molecule and (b) immobilizing each immune effector molecule to a detection particle specific for the immune effector molecule; and
(c) measuring the presence or concentration of the reporter in a multiplex assay, wherein the presence or concentration of the reporter corresponds to the presence or concentration of each immune effector molecule in the biological sample.

2. The method of claim 1 wherein the biological sample is porcine.

3. The method of claim 1 or claim 2 wherein the at least seven immune effector molecules are chosen from the group consisting of IL-1β, IL-4, IL-6, IL-8, IL-10, IL-12, IFN-α, IFN-γ, and TNF-α.

4. The method of claims 1-3 wherein the biological sample is serum.

5. The method of claims 1-4 wherein the concentration of the at least seven immune effector molecules in the biological sample is in the picomolar range.

6. The method of claims 1-5 wherein the capture particle is a monoclonal antibody.

7. The method of claim 6 wherein the monoclonal antibody is bound to a solid phase support.

8. The method of claim 7 wherein the solid phase support is a microsphere.

9. The method of claims 1-8 wherein the detection particle is a monoclonal antibody.

10. The method of claims 1-9 wherein the detection particle is biotinylated.

11. The method of claims 1-10 wherein the biological sample is collected from a subject prior to vaccination.

12. The method of claims 1-10 wherein the biological sample is collected from a subject subsequent to vaccination.

13. The method of claims 1-12 wherein incubation with the capture particle and incubation with the detection particle is done sequentially.

14. The method of claims 1-13 further comprising a wash step between incubation with the capture particle and incubation with the detection particle.

15. A method of determining immunity status of a subject, comprising:
(a) detecting the concentration of at least seven immune effector molecules in a biological sample of interest in a multiplex assay, and
(b) determining immunity status of a subject based on concentrations of the at least seven immune effector molecules in the biological sample of interest.

16. The method of claim 15 wherein the immunity status of the subject is a measurement of the immunity to porcine reproductive and respiratory syndrome virus (PRRSV).

17. A kit comprising reagents for simultaneously detecting at least seven immune effector molecules in a porcine biological sample of interest.

18. The kit of claim 17 wherein the at least seven immune effector molecules are chosen from the group consisting of IL-1β, IL-4, IL-6, IL-8, IL-10, IL-12, IFN-α, IFN-γ, and TNF-α.

19. The kit of claim 17 and claim 18 wherein the reagents comprise a monoclonal capture antibody and a monoclonal detection antibody specific for at least seven immune effector molecules.

20. The kit of claim 19 wherein the reagents further comprise a buffer.

21. A method of detecting the presence or concentration of a plurality of immune effector molecules in a biological sample, comprising:
(a) incubating porcine serum under suitable conditions with nine monoclonal capture antibodies, wherein the monoclonal capture antibodies are immobilized on a microsphere bead, further wherein each monoclonal capture antibody is specific for an immune effector molecule consisting of IL-1β, IL-4, IL-8, IL-10, IL-12, IFN-α, IFN-γ, or TNF-α;
(b) forming a microsphere bead/monoclonal capture antibody/immune effector molecule complex;
(c) washing the microsphere bead/monoclonal capture antibody/immune effector molecule complex in a buffer;
(d) incubating the micro sphere bead/monoclonal capture antibody/immune effector molecule complex under suitable conditions with nine biotinylated monoclonal detection antibodies, wherein each monoclonal detection antibody is specific for one microsphere bead/monoclonal capture antibody/immune effector molecule complex;
(e) forming a microsphere bead/monoclonal capture antibody/immune effector molecule/monoclonal detection antibody complex;
(f) washing the microsphere bead/monoclonal capture antibody/immune effector molecule/monoclonal detection antibody complex in a buffer;
(g) incubating the microsphere bead/monoclonal capture antibody/immune effector molecule/monoclonal detection antibody complex with strepavidin-R-phycoerthrin,
(h) washing the microsphere bead/monoclonal capture antibody/immune effector molecule/monoclonal detection antibody/strepavidin-R-phycoerthrin complex in a buffer; and
(i) detecting the presence or concentration of a phycoerthrin fluorescence by flow cytometry, wherein the presence or concentration of phycoerthrin fluorescence corresponds to the presence or concentration of each immune effector molecule in the porcine serum.
